(19) United States
(12) Patent Application Publication
(10) Pub. No.: US 2020/0020414 A1
(43) Pub. Date: Jan. 16, 2020

(54) IN-SILICO METHOD TO IDENTIFY COMBINATORIAL PROTEINS AS IMMUNE-STIMULATORS AGAINST LEISHMANIASIS

(71) Applicant: COUNCIL OF SCIENTIFIC & INDUSTRIAL RESEARCH, New Delhi (IN)

(72) Inventors: Ram Rup Sarkar, Pune (IN); Piyali Ganguli, Pune (IN); Saikat Chowdhury, Pune (IN)

(21) Appl. No.: 15/776,008

(22) PCT Filed: Nov. 9, 2016
(86) PCT No.: PCT/IN2016/050390

§ 371 Date: May 14, 2018

(30) Foreign Application Priority Data
Nov. 12, 2015 (IN) 3691/DEL/2015

(51) Int. Cl. G16B 5/00; G16B 20/00

(52) U.S. Cl. CPC G16B 5/00; G16B 20/00

(57) ABSTRACT

The present invention discloses a combination of proteins influencing the survival of the Leishmania species inside the human cell and a process for regulating the expression the combination of proteins. Further, the present invention relates to the regulation of the combination of proteins to serve as immuno-stimulators to treat leishmaniasis.

### FIELD OF THE INVENTION

The present invention relates to an in-silico method to identify combinatorial proteins as immune-stimulators against Leishmaniasis.

Further, the present invention relates to the said combinatorial proteins influencing the survival of the Leishmania species inside the human cell and a process for regulating the expression of a combination of proteins to serve as immuno-stimulators to treat Leishmaniasis.

### BACKGROUND OF THE INVENTION

Cell-mediated immunity (CMI) is an immune response characteristic to the human host system for combating infections caused due to invasion by intra-cellular pathogens, primarily involving interactions between phagocytic Antigen Presenting Cells (APC's) and T-cell lymphocytes. These interactions lead to activation of a series of intra-cellular and inter-cellular biochemical signaling processes, which culminates into synthesis of certain diffusible effector molecules including proteins and microbicidal molecules aiding in the clearance of the pathogen causing the disease (P. Kaye et al Nat. Rev. Micro. 9 (2011) 604-15). However, activities of this defense mechanism are severely compromised during Leishmaniasis caused due to protozoan parasites of the genus Leishmania.

The Leishmania parasite is transmitted to humans through infected bites of Phlebotomine sand flies during their blood meal. Leishmania exist in two basic body forms: the amastigote, the intracellular form in the vertebrate host, and the promastigote, the extracellular form in the sandfly vector (Phleobotomus spp. and Lutzomyia spp.).The promastigote form of Leishmania on gaining entry into a human host is engulfed by APC including macrophages and dendritic cells to form a phagolysosome, where it differentiates into its amastigote form and takes control of the entire cellular machinery such that immuno-competency of immune cells is reduced, thereby hindering body's natural parasite clearance process (M. Olivier et al Clin. Microbiol. Rev. 18 (2005) 293-305). The surface molecules produced by Leishmania, such as, lipophosphoglycan (LPG), glycoprotein 63 (GP63) and the elongation factor EF1-alpha directly or indirectly activate a series of phosphatases inside human Antigen Presenting cells (APCs), that leads to de-phosphorylation and deactivation of important signaling molecules inside the host cell (M. T. Shio et al J. Trop. Med. (2012) 819512). LPG molecules secreted inside APC's serve as antigens and are presented to the surrounding T-lymphocytes to elicit either healing or non-healing immune response, depending on the parasite load and the host immunity(J. N. Menon et al Eur. J. Immunol. 28 (1998) 4020-4028).The healing response is obtained in case of low parasitic load, in which pronounced Type-I helper T-cell (Th1) response occurs due to up-regulation of Th1 cytokines, such as Interferon Gamma (IFN γ) from stimulated T-cells, and thus naturally clears pathogens from the system. A higher pathogen load results in the rise of a non-healing response in which an up-regulation of the Th2 cytokines (e.g. IL10) is observed, that favors persistence of Leishmania. During non-healing response, production of protective Th1 cytokines, such as IL12, and microbicidal molecules, such as nitric oxide are also down-regulated, thus creating an immune-suppressed condition suitable for further progression of Leishmaniasis (D. Liu, and J. E. Uzonna, Front Cell Infect Microbiol. 2 (2012) 83).

Experimentally these types of immune responses are proven to be elicited in humans diagnosed with all forms of Leishmaniasis, viz. cutaneous, muco-cutaneous and visceral (D. McMahon-Pratt and J. Alexander, Immunol. Rev. 201 (2004) 206-24). Hence, the general therapeutic strategy adopted for Leishmaniasis treatment is primarily aimed to expedite the process of parasite clearance for faster healing by stimulating Th1 or healing response.

Chemotherapeutic drugs, such as pentavalent antimonials, liposomal amphotericin B have been shown to be useful to reduce the dermal lesions and probability of further destructive mucosal inflammations and visceral infections in cutaneous leishmaniasis. However, successive clinical studies have shown that these drugs are also associated with adverse side effects, nausea, intense headache, diarrhea, musculoskeletal and abdominal pain. In several cases, relapse of Leishmaniasis and development of resistant strains are also reported after regular antibiotic use, which necessitates development of more efficient treatment protocols with higher clinical efficacy (S. L. Croft et al Clin. Microbiol. Rev. 19 (2006) 111-26).

Although immunotherapeutic strategies involving administration of exogenous interferon Gamma is found effective in suppressing Leishmaniasis, high production of IL10 during early stage of infection often suppresses IFN-γ activity, thereby hindering Nitric oxide (NO) production and pathogen clearance.

Based on experimental investigations of mathematical models, M. Mol et al (Biochem. Biophys. Acta, 1840 (2014) 71-79 and Syst. Synth. Biol. 7 (2013) 185-195) have also proposed potential pharmacological targets by identifying CD14, Tumor Necrosis factor (TNF) and Epidermal Growth factor receptor (EGFR) signalling which can be linked through crosstalk points. Pro-inflammatory response may be achieved through EGFR in leishmaniasis. However, identification of these effector molecules have not provided a direct role in regulation of Th1 and Th2 responses, since regulation of Th cells aid in termination of parasite growth inside host cell.

A research study by Castellano L R et al published in Hum Immunol. 2009 June; 70(6):383-90 suggests the presence of a mixed Th1/Th2 response during active disease and that clinical cure is associated with a sustained Th1 response characterized by elevated IFN-γ levels and down-modulation of IL-4 and IL-10 production.

Cytokine therapy is the most widely used therapy in the treatment of Leishmaniasis. Cytokine therapy administered by exogenous injection of IFN γ and suppression of IL10 to eradicate Leishmania pathogens in macrophage cells have been proposed (L. Albergante et al, PLoS Comput. Biol. 9 (2013) e1003334). However, IFN γ is a pro-inflammatory molecule and also has a short half-life time, therefore its repeated administration into the body at regular intervals of time is required resulting in harmful consequences. The inhibitory effect of IL10 protein, which is overexpressed during infection, increases the susceptibility of the host to the disease by inhibiting the effects of IFN γ treatment and often blocking synthesis of NO thereby preventing effective anti-Leishmania immunity. Hence, to evade these drawbacks, implementation of enhanced therapeutic strategies, by identifying novel drugs, drug target molecules and immuno-stimulators is required. However, in order to develop an effective immunotherapeutic strategy, it is important to understand the unexplored Th1/Th2 duality in Leishmaniasis to identify regulators through which Th1/Th2 switching behavior can be effectively controlled. Inadequate knowledge of this mechanism of Leishmania species invasion inside the host immune system is the key reason for low success in devising an effective cure of Leishmaniasis. In order to overcome this short-coming, it is necessary to gain insight into the precise mechanism of regulation of Th1/Th2 activity by which Leishmania antigen molecules takes control of the host cell's signaling processes.

Therefore, through in-silico modelling methods, the present inventors have devised a therapeutic strategy to eliminate Leishmania from host cells by identifying novel combinations of immuno-stimulators that facilitate stimulation of Type-I T-helper cells and also provide simultaneous up-regulation of NO production. The combination of protein molecules can serve as potent immuno-stimulators targeting of which may bypass inhibitory activities of Leishmania to yield an effective anti-Leishmania immune response and expedite the process of parasite clearance from the system.

### OBJECTS OF THE INVENTION

An object of the present invention is to provide an in-silico method to identify combinatorial proteins as immune-stimulators against leishmaniasis. It further provides a regulatory mechanism that controls the survival of Leishmania species in human host system by in-silico modeling, by focusing on three important aspects of Leishmania immunobiology
(a) effect of Leishmania infection on gene expression or the protein activation pattern in Antigen presenting cells (APC) and microbicidal activities,
(b) effect of Leishmania infection on the T-cell gene/protein expression pattern at the molecular level and their influence in pathway level to identify the molecular routes by which Leishmania inhibits T-cell functions, and
(c) identification of immunostimulators that could serve as a regulatory switch to modify Th1/Th2 dynamics towards healing Th1 response and simultaneously enhance the NO production in order to accelerate the parasite clearance from the host cell.

Another object of the present invention is to provide regulatory mechanisms that control the survival of Leishmania species in human host system by in-silico modeling, comprising identification of immuno-stimulators that serve as a regulatory switch to modify Th1/Th2 dynamics towards healing Th1 response and to simultaneously enhance the nitric oxide (NO) production in order to accelerate Leishmania parasite clearance from the host cell.

Another object of the invention is to provide a process for regulation of a combination of proteins to modify the protein expression pattern leading to the expedited clearance of Leishmania parasites from the host system.

### SUMMARY OF THE INVENTION

The present invention provides an in-silico model comprising a signaling network of interactions between antigen molecules of Leishmania and the APC (antigen presenting cells) and T-cell pathway.

The entire signaling network of Leishmania-APC-T-cell pathway model consists of a total of 293 nodes/molecules, which include 82 APC molecules, 206 T-cell molecules, and 5 Leishmania related molecules, involved in more than 400 protein-protein interactions. The intra-cellular signaling cascades considered for modeling the APC and the T-cell consists of the major co-receptor signaling pathways, the cytokine pathways, TLR pathways, etc. that play a pivotal role in regulating the outcome of the immune cell's functional responses.

In the most preferred aspect, the present invention provides a combination of immuno-stimulators selected from the group consisting of Toll like receptor-2 (TLR-2) and Toll like receptor 3 (TLR-3) in Antigen presenting cells (APC's) and Src Homology 2 phosphatase (SHP2) in T-cells, Mitogen activated protein kinase phosphatase (MKP) and SHC in T-cells for simultaneously upregulating TH1 response and nitric oxide (NO) production, and downregulating TH2 response to expedite the clearance of Leishmania pathogen from an infected host cell.

The present invention provides a simultaneous up-regulation of the healing Th1 response and stimulation of Nitric Oxide (NO) production from the Antigen presenting cells (APCs), and down-regulation of the non-healing Th2 response by regulating the aforesaid combinations of protein molecules that elicit an anti-Leishmania immune response. Accordingly, the present invention provides increasing activity of Toll like receptor-3 molecules (TLR3) for eliciting NO synthesis to inhibit Leishmania growth, and reducing activity of Toll like receptor-2 molecules (TLR2) to inhibit an anti-Leishmania immune response.

In another aspect, the present invention provides a process for up regulating nitric oxide (NO) production and TH₁ response and down regulating TH₂ response simultaneously in a mammalian host cell during Leishmania infection by targeting protein groups selected from the group consisting of TLR3 in APC's and SHP2 (in T-cell) or TLR3 (in APC), MKP and SHC in T-cells.

Accordingly, the present invention provides an immunotherapeutic process comprising regulating at least one of the combinations of proteins/immuno-stimulators to expedite the process of clearance of Leishmania pathogen from the host cell: (i) up regulation of TLR3 and down regulation of SHP2_T and (ii) up regulations of TLR3, MKP_T and down regulation of SHC_T, are considered as better than solitary TLR2 inhibition.

Further, the present invention provides that Leishmania infection induces an up-regulation of IFN beta production from the APC that may lead to an up-regulation of the RAP1 and SOCS3 proteins inside the T-cell, the potential inhibitors of MAPK and JAK-STAT signaling pathways respectively, via the TYK2-mediated pathway.

The present invention provides for the regulation of combinations of proteins as potential immune-modulators to promote healing response, i.e. Th1cell response during leishmaniasis.

In yet another aspect, the present invention provides a process for up regulating nitric oxide (NO) production and TH₁ response and down regulating TH₂ response simultaneously in a mammalian host cell during Leishmania infection comprising;
(i) up regulation of TLR3 is obtained by administering agonist Rintatolimod,
(ii) up regulation of MKP is obtained by administering agonist JWHO15,
(iii) down regulation of SHP2 is obtained by administering Actinomycin D, and
(iv) down regulation of SHC is obtained by administering 8-hydroxy-7-(6-sulfo naphthalene-2-yl)diazenyl-quinoline-5-sulfonic acid.

In one more aspect, the present invention provides a method for treating leishmaniasis comprising regulating at least one of the combinations selected from;
(i) up regulating TLR3 and down regulating of SHP2, and/or
(ii) up regulating TLR3, MKP and down regulating SHC,

wherein the said process comprises
(a) up regulating TLR3 by administering agonist Rintatolimod,
(b) up regulating MKP by administering agonist JWHO15,
(c) down regulating SHP2 by administering Actinomycin D, and
(d) down regulating SHC by administering 8-hydroxy-7-(6-sulfo naphthalene-2-yl)diazenyl-quinoline-5-sulfonic acid.

### BRIEF DESCRIPTION OF ACCOMPANYING DRAWINGS

FIG. 1 depicts a simplified pathway diagram showing Leishmania-APC and T-cell interaction. The figure shows juxtacrine and paracrine regulations between different cells. The Leishmania antigen molecules are shown in orange. The cytoplasmic and nuclear proteins of the APC and T-cell are color coded as blue and peach respectively. The target molecules that are produced as output by the two cells are colored green (for protein) and deep-pink (for non-protein molecules);

FIG. 2 depicts time-course expression profile of APC output molecules namely, c_FOS, IFN_BETA, IL1_ALPHA, IL1_BETA, IL10, IL12, INOS, IP10, NO and TNF_ALPHA found in infected, uninfected and experimental conditions. The validation was performed by comparing the expression levels of the infected situations (shown in red) with the microarray experimental data (black diamond);

FIG. 3 depicts expression profile of T-cell and APC during asynchronous simulation, FIGS. 3(a) and (b) show expression of the APC output molecules in the uninfected and infected scenarios respectively; FIGS. 3(c) and (d) Expression of the T-cell output proteins in the uninfected and the infected scenarios respectively;

FIG. 4 depicts expression profile of 20 T-cell proteins which show significant de-regulation in Mann-Whitney U test. The heat maps depict the protein expression pattern of the T-cell signaling proteins under (a) Uninfected scenario (control); (b) Infected scenario. Significant changes in the expression dynamics are observed for these proteins under these two conditions, which clearly show the effect of Leishmania antigens in the regulation of T-cell signaling events;

FIG. 5 depicts the response dynamics of Th1 , Th2 and Nitric Oxide in uninfected, infected and in different treatment scenarios. (a) uninfected; (b) infected; (c) IL12 on; (d) IFN_GAMMA_T on; (e) MKP_T on; (f) TLR3 on; (g) SHP2_T off; (h) SHC_T off; (i) TLR2 off; (j) TLR3 on and SHP2_T off; (k) TLR3, MKP_T on and SHC_T off;

FIG. 6 depicts T-cell pathways de-regulated during leishmaniasis. The schematic diagrams provide following (a) Infected APC produces high amount of IFN_Beta, which in turn up-regulates production of SOCS3 and RAP1 proteins that have negative regulatory effects on its down-stream JAK-STAT and MAPK pathways; (b) Infected APC inhibits production of IL12 cytokine which results in up-regulation of IL4, IL5 and IL6 cytokine secretion from T-cell by regulating JAK/STAT and IFNγ_T protein production. Green upward arrow—protein expression up-regulated; Red downward arrow—protein expression down-regulated; Black arrow—activation; Red arrow—inhibition.

### DETAILED DESCRIPTION OF THE INVENTION

The invention will now be described in detail in connection with certain preferred and optional embodiments, so that various aspects thereof may be more fully understood and appreciated.

In the description for the purposes of the present invention ‘helper T cells’ are referred herein in the abbreviated form as ‘TH’ or ‘Th’ cells or immune response and shall be denoted to both type 1 and type 2 T helper cells as well as to immune responses.

Further, TLR refers to Toll like receptors and is referred to as TLR 2 and TLR 3 in the present specification.

The combination of protein molecules addressed herein refers to the combination of immuno-stimulators or immuno-modulators for the purposes of the present invention.

The present inventors have provided a combination of immuno-stimulators to clear Leishmania pathogens from the body without adverse side effects, by stimulating type-I T-helper cells and a simultaneous upregulation of NO production by regulating the expression of an immuno-stimulator or combination of immuno-stimulators.

In the most preferred embodiment, the present invention provides a combination of immuno-stimulators selected from the group consisting of Toll like receptors-2 (TLR-2) and Toll like receptor 3 (TLR-3) in Antigen presenting cells (APC's) and Src Homology 2 phosphatase (SHP2) in T-cells or TLR3 in APC's, Mitogen activated protein kinase phosphatase (MKP) and SHC in T-cells which when regulated expedite the clearance of Leishmania pathogen from an infected host cell.

In order to arrive at the present combination of protein immuno-stimulators, the present inventors have provided a manual reconstruction of a cell signalling pathway map of Leishmania infected APC and a normal CD4+ T cell (helper T cell), considering the important physical interactions and the cross-talks by the secreted diffusible molecules between the two cells.

Accordingly, an in-silico model comprising a signaling network of interactions between antigen molecules of Leishmania and the APC (antigen presenting cells) and T-cell pathway is provided herein.

In an embodiment, the present invention provides an in-silico method to identify combinatorial proteins as potent immune stimulators to treat leishmaniasis comprising steps:
(i) reconstructing Leishmania-APC-T-cell pathway model by integrating inter-cellular and intra-cellular signalling events occurring between APC (Antigen Presenting cells) and T cell during Leishmania invasion;
(ii) simulating the Leishmania-APC-T-cell pathway model reconstructed in step (i) by AND, OR and NOT logical gates in infected and uninfected scenarios to obtain immune responses;
(iii) validating the immune responses as simulated in step (ii) with published literatures to confirm their acceptability and authenticity to obtain validated immune responses;
(iv) perturbing the validated immune responses of step (iii) to identify immuno-stimulating proteins each from APC and T-cell respectively;
(v) performing single in silico knock in/knock out mutation of the proteins identified in step (iv) to obtain in silico knock in/knock out mutated proteins;
(vi) recognizing combination of the mutated proteins as potent immunostimulators post in silico mutation analysis in step (v) and devising their regulation to yield an effective anti-leishmania response.

In another embodiment of the present invention there is provided the in-silico method to identify combinatorial proteins, wherein the combinatorial proteins comprises of three T-cell and two APC molecules.

Another embodiment of the present invention provides in-silico method to identify combinatorial proteins, wherein the T-cell molecules are selected from the group consisting of MKP_T, SHP2_T, and SHC_T.

In yet another embodiment of the present invention there is provided the in-silico method to identify combinatorial proteins, wherein the APC molecules are TLR3 and TLR2.

Still another embodiment of the present invention provides the in-silico method to identify combinatorial proteins, wherein the Leishmania-APC-T-cell pathway model comprises 293 nodes, 82 APC molecules, 206 T-cell molecules and 5 Leishmania related molecules.

An embodiment of the present invention provides the in-silico method to identify combinatorial proteins, wherein the simulating the model in step (ii) results in three phenotypic functions “TH_1_response” (Eq. 1), “TH_2_response”(Eq. 2) and “NO_response (Eq. 3).

In another embodiment of the present invention there is provided the in-silico method to identify combinatorial proteins, wherein the in silico knock in/knock out mutated proteins of step (v) are assigned ON/TRUE and OFF/FALSE to up regulate or down regulate the phenotypic functions identified in the present application.

Still another embodiment of the present invention provides the in-silico method to identify combinatorial proteins, wherein the combination of immuno-stimulators of step (vi) is selected from the group consisting of Toll like receptor-2 (TLR-2) and Toll like receptor 3 (TLR-3) in Antigen presenting cells (APC's), Src Homology 2 phosphatase (SHP2) in T-cells, or Mitogen activated protein kinase phosphatase (MKP) and SHC in T-cells, for simultaneously regulating nitric oxide (NO) production, TH1 immune response and TH2 response to expedite clearance of Leishmania pathogen from an infected host cell.

Yet another embodiment of the present invention provides the in-silico method to identify combinatorial proteins, wherein a process to increase NO production and TH1 immune response and inhibit TH2 response simultaneously in a Leishmania infected host cell comprises regulating at least one combination selected from:
(a) up regulation/stimulation of TLR3 in APC and down regulation/inhibition of SHP2 in T-cell; and
(b) up regulations/stimulation/activation of TLR3 in APC, MKP in T-cell and down regulation/inhibition of SHC in T-cell.

Another embodiment of the present invention provides the use of the combinatorial proteins to treat cutaneous leishmaniasis.

Yet another embodiment of the present invention provides the use of the combinatorial proteins to control Th1/Th2 immune response during leishmanial infection and to eliminate the parasite from the system.

An embodiment of the present invention provides the method for treating leishmaniasis comprising regulating at least one of the combinations of immune-stimulators of the present invention, wherein the combination is selected from:
(i) up regulating TLR3 and down regulating of SHP2, and
(ii) up regulating TLR3, MKP and down regulating SHC,
wherein said method comprises:
(a) up regulating TLR3 by administering agonist Rintatolimod,
(b) up regulating MKP by administering agonist JWHO15,
(c) down regulating SHP2 by administering Actinomycin D, and
(d) down regulating SHC by administering 8-hydroxy-7-(6-sulfo naphthalene-2-yl)diazenyl-quinoline-5-sulfonic acid.

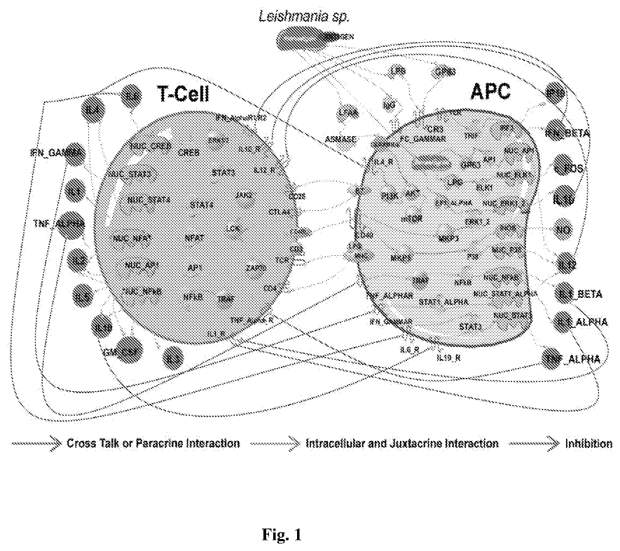

In the present model (FIG. 1), three functions, viz. “TH_1_response”, “TH_2_response” and “NO_response”, reflecting the type of T-cell responses elicited and production of NO from APC in response to Leishmania infection is represented by equations: Eq. 1, 2 and 3. The equations are defined as follows:

TH_1_response*=IL2_T AND GM_CSF_T AND TNF_ALPHA_T AND IFN_GAMMA_T (Eq. 1)

TH_2_response*=IL4_T AND IL5_T AND IL6_T AND IL10_T (Eq. 2)

NO_response*=NO (Eq. 3)

FIG. 1 shows physical binding of T-cell and APC receptors/co-receptors with their corresponding ligands and subsequent activation mechanism of downstream proteins in both cells.

The present model considers activation of TLR proteins, present in the APC membrane, which activate their downstream proteins, which in turn diverge into important signaling routes such as the RAS-RAF mediated MAPK pathway (Mitogen activated protein kinases), canonical and non-canonical NFKB pathway (Nuclear factor kappa-light-chain-enhancer of activated B cells), JAK-STAT pathway (JAK-STAT system comprises of two main components: a receptor, Janus kinase (JAK) and Signal Transducer and Activator of Transcription (STAT)), PI3K-PLC Gamma pathway, JNK (c-Jun N-terminal kinases) pathway and lead to the activation of several transcription factors selected from ERK1_2, NFKB, NFAT, AP1, STAT in the nucleus, that in due course, singly or in combination with other transcriptional co-factors initiates protein production (S. Bhardwaj et al J. Biomed. Biotechnol. (2010) 109189). Proteins principally cytokines, growth factors and cell cycle proteins synthesized at the end of the cascade, in response to pathogenic invasion, manifest externally in the form of a change in the cellular behavior, herein referred to as a ‘phenotypic response’ viz. the Th1-Response, Th2-Response and NO-Response (Eq. 1, 2 and 3).

The present model predicts the phenotypic responses using Eq. 1, 2 and 3 in various treatment scenarios using several gene knock-in and knock-out experiments created in-silico by trying different combinations of the protein molecules.

In a preferred embodiment, the present invention identifies three T-cell molecules selected from MKP (MAP Kinase Phosphatases), SHP2 (also termed as Tyrosine-protein phosphatase non-receptor type 11 (PTPN11) and SHC (Src Homology 2); and two APC molecules selected from TLR3 and TLR2 having important role in Leishmania pathogen clearance. While MKP, SHC and TLR3 have a positive role in eliciting an anti-Leishmania response, SHP2 and TLR2 exhibit a negative role for the same. The agonist and the antagonists of these target molecules have been listed in the table 1

TABLE 1

| Targets | Antagonist/Agonist | Reference |
|---|---|---|
| TLR2 | Antagonist- C16H15NO4 | P. Mistry et al. ProcNatlAcadSci USA |
| | | 112(2015) 5455-60. |
| TLR3 | Agonist- polyIC₁₂U | C. F. Nicodemus and J. S. Berek |
| | (Rintatolimod) | Immunotherapy 2(2010) 137. |
| MKP | Agonist- JWH015 | E. A. Romero-Sandoval et al. Mol Pain |
| | | 5(2009) 25. |
| SHC | Antagonist- PP2 | J. E. Brown et al. J Neurosci 30(2010) 5242- |
| | Inhibitor of Shc/Grb2 | 52. |
| | interaction- actinomycin D | H. K. Kim et al Life Sci 78(2005) 321-8. |
| SHP2 | Antagonist- 8-hydroxy-7-(6- | L. Chen et al. MolPharmacol 70(2006) 562-70. |
| | sulfonaphthalen-2-yl)diazenyl- | |
| | quinoline-5-sulfonic acid (NSC- | |
| | 87877) | |

The entire signaling network of Leishmania-APC-T-cell pathway model consists of a total of 293 nodes, including 82 APC molecules, 206 T-cell molecules, and 5 Leishmania related molecules, involved in more than 400 protein-protein interactions. The intra-cellular signaling cascades considered for modeling APC and T-cell consists of the major co-receptor signaling pathways, the cytokine pathways, TLR pathways, etc. that play a pivotal role in regulating the outcome of the immune cell's functional responses.

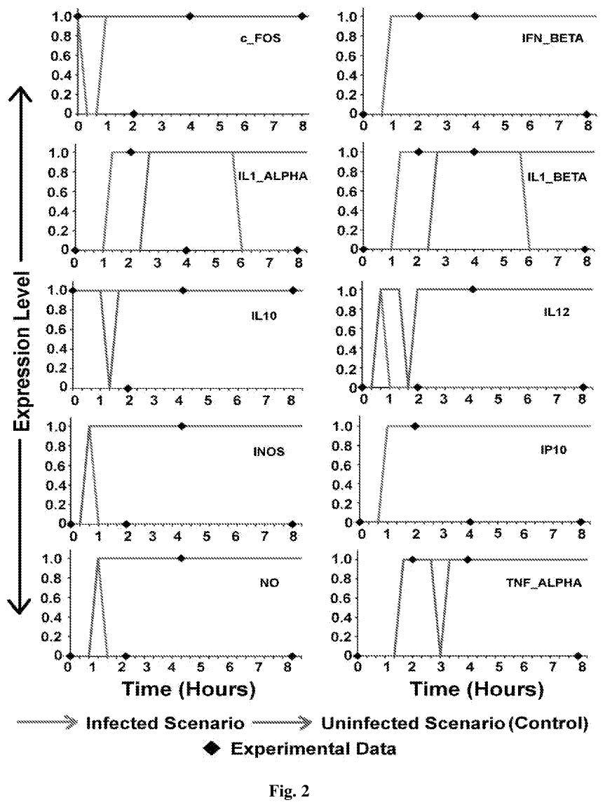

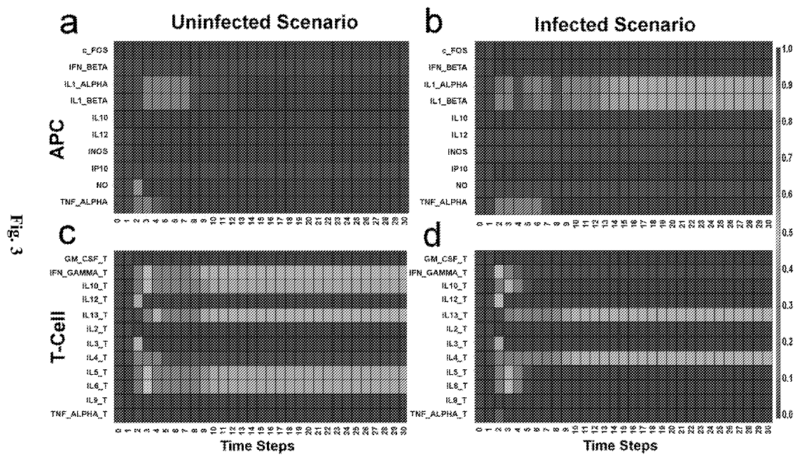

Further, a comparison of the infected (Leishmania) and uninfected scenarios to bring out the effect of Leishmania infection on the expression of output molecules in both APC and the T-cell (FIGS. 2, 3) is studied. Accordingly, Leishmania infection down-regulates production of protective cytokines, such as IL12, IL1α and IL1β, and microbicidal molecules, such as NO, and simultaneously up-regulates the production of chemokine, IP10. The simulation also reveals production of cytokine IFNβ which is up-regulated in the infected scenario.

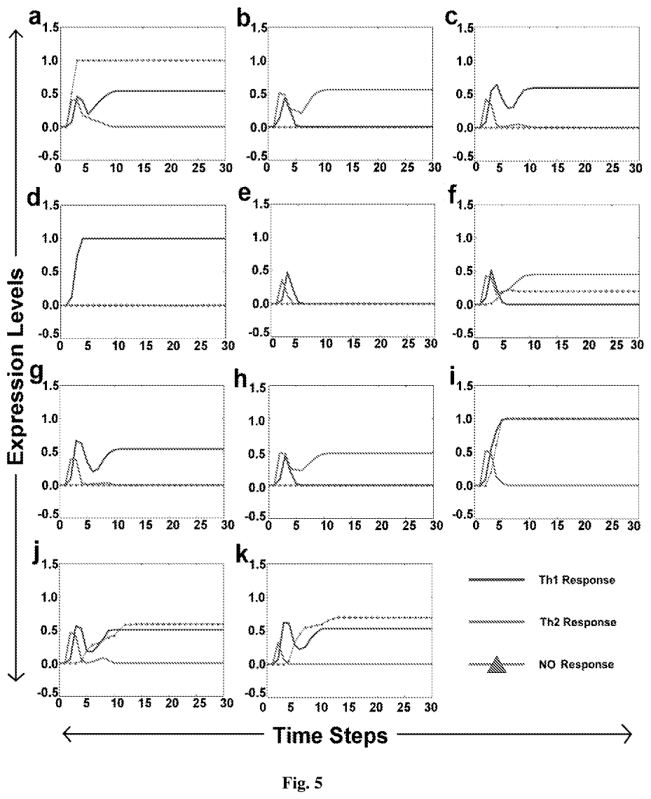

The T-cell expression profile shows that during Leishmania infection, interleukin molecules viz. IL10_T, IL4_T, IL5_T and IL6_T, get up-regulated, while expression of IFN γ get down-regulated (FIGS. 3c, d). The higher production of proteins, such as IL10_T and IL4_T and repression of IFN γ _T synthesis, produces conditions that favor Leishmania survival, and skews Th1/Th2 dynamics towards a non-healing response, i.e. the Th2 response (FIG. 5b).

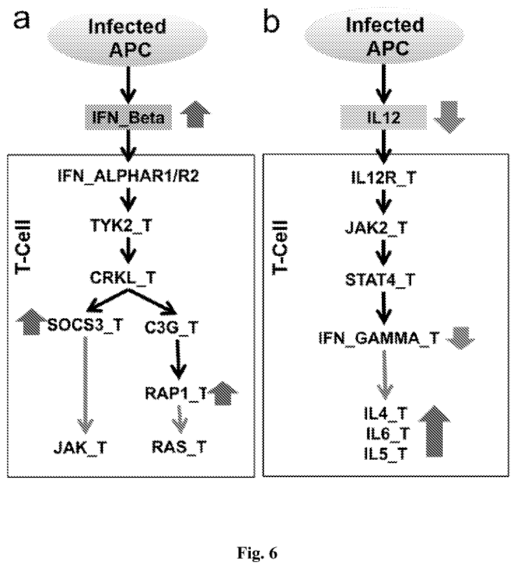

Identified from simulation, the regulatory mechanisms of the signaling cascades are presented in FIG. 6. Accordingly, Leishmania infection increases production of the protein IFNβ (green upward arrow) and suppresses IL12 (red downward arrow) from the APC. IFNβ diffuses and interacts with their corresponding receptors on the T-cell thereby enhancing the activation of its downstream TYK2 molecule (black arrow) inside the T-cell.

Through this analysis, the possible role of L. major infection in modulating the T-cell behavior at the pathway level, and infer that the pathogen up-regulates the molecules involved in the TYK-CRKL-C3G pathway was performed. Eventually, it enhances the production of SOCS3 and RAP1 proteins in the T-cell (FIG. 6a), two potential negative regulators of JAK-STAT and the RAS mediated MAPK pathways respectively (red arrow), which divulges the probable harmful effects of the high levels of IFNβ production from the APC that is known to occur during Leishmania infection.

Moreover, it can be observed that in T-cell (FIG. 6b), Leishmania down-regulates JAK2-STAT4 pathway by inhibiting synthesis of IL12 cytokine, which results in down-regulation of IFN-γ production (red downward arrow) and a consequent increase in the IL4_T, IL5_T and IL6_T expression (green upward arrow). These findings of changes occurring at the pathway level have further identified the key regulators that can act as potential immuno-stimulators during the infection.

In view of the use of immunotherapies employing IL12 treatment and IFN_GAMMA_T treatment, the present inventors have simulated the effect of these two (FIGS. 5c & d) strategies, and have observed that although they are able to enhance Th1 response and reduce Th2 response, they fail to induce NO response, vital to the elimination of Leishmania pathogen.

Hence, to devise a successful combinatorial immunotherapy, which can bypass the inhibitory effects of immune-suppressive molecules, various molecules that directly or indirectly influence the de-regulated T-cell pathways (i.e. JAK2-STAT4 pathway and the TYK2-mediated IFN_BETA pathways) and TLR molecules of the Antigen Presenting Cell are selectively knocked-in and knocked-out separately and then in combination (Table 2).

Thereafter, a set of minimal combinations of protein molecules are identified that act as a regulatory switch to control Th1/Th2 response and also effectively enhance an anti-Leishmania response (Table 2).

In another preferred embodiment, the present invention provides increasing activity of Toll like receptor-3 molecules (TLR3) for eliciting NO synthesis to inhibit Leishmania growth, and reducing activity of Toll like receptor-2 molecules (TLR2) to inhibit an anti-Leishmania immune response.

In yet another preferred embodiment, the present invention provides an immuno-stimulator combination comprising TLR3, MKP_T and SHC_T to skew Th1/Th2 response in favor of healing Th1 response and elicit nitric oxide (NO) synthesis, wherein TLR3, MKP_T in said combination when up-regulated and SHC_T when down-regulated clears the Leishmania pathogen from the host system.

Accordingly, the present invention provides a process for up regulating nitric oxide (NO) production and TH₁ response and down regulating TH₂ response simultaneously in a mammalian host cell during Leishmania infection by targeting protein groups selected from the group consisting of TLR3 (in APC) and SHP2 (in T-cell) or TLR3 (in APC), MKP and SHC (in T-cell).

The an immunotherapeutic process comprising regulating at least one of the combinations of proteins/immuno-stimulators to expedite the process of clearance of Leishmania pathogen from the host cell: (i) up regulation of TLR3 and down regulation of SHP2_T and (ii) up regulations of TLR3, MKP_T and down regulation of SHC_T, are considered as better than solitary TLR2 inhibition.

In the present invention, inhibition of TLR2 is considered to be a useful strategy to up-regulate Th1 and NO response (FIG. 5i). On the other hand TLR3 has a positive role to play in Leishmania treatment and is a positive regulator of NO production (FIG. 5f). Although TLR2 inhibition alone is sufficient to drastically enhance Th1 response and NO production (FIG. 5i), TLR3 activation requires a synergistic inhibition of SHP2_T molecule, a phosphatase that inhibits activity of JAK-STAT pathway, to gain desired anti-Leishmania response (FIG. 5j). Further, MAPK phosphatase (MKP_T) when up-regulated may inhibit non-healing Th2 response (FIG. 5e). However, MKP_P and TLR3 up-regulation when combined with inhibition of the adapter molecule SHC_T, a positive regulator of the MAPK cascade, can act as a useful combinatorial target in Leishmaniasis treatment (FIG. 5k). Nevertheless to combat Leshmaniasis, it may be noted here that since Th1 subset of helper T-cells produces inflammatory cytokines, a constant high Th1 response may often be undesirable in order to avoid harmful side-effects.

Therefore the combinations: (i) up regulation of TLR3 and down regulation of SHP2_T and (ii) up regulations of TLR3, MKP_T and down regulation of SHC_T, are considered as better immunotherapeutic strategies than solitary TLR2 inhibition.

In an alternative embodiment, the expression of genes encoding the proteins selected from the three T-cell molecules i.e. MKP (MAP Kinase Phosphatases), SHP2 (also termed as Tyrosine-protein phosphatase non-receptor type 11 (PTPN11) and SHC; and two APC molecules selected from TLR3 and TLR2 are regulated so as to obtain the clearance of Leishmania pathogen from an infected host cell.

In one preferred embodiment, the present invention provides a process for up regulating nitric oxide (NO) production and TH₁ response and down regulating TH₂ response simultaneously in a mammalian host cell during Leishmania infection comprising;
(i) up regulation of TLR3 is obtained by administering agonist Rintatolimod,
(ii) up regulation of MKP is obtained by administering agonist JWHO15,
(iii) down regulation of SHP2 is obtained by administering Actinomycin D, and
(iv) down regulation of SHC is obtained by administering 8-hydroxy-7-(6-sulfo naphthalene-2-yl)diazenyl-quinoline-5-sulfonic acid.

In one embodiment, the present invention provides a method for treatment of leishmaniasis, wherein a therapeutically effective agonist to proteins TLR3 and MKP_T and a therapeutically effective antagonist to TLR2, SHP2_T and SHC_T may be administered to a subject in need thereof (see Table 1 for the probable list of agonist and antagonist molecules).

Accordingly, the present invention provides a method for treating leishmaniasis comprising regulating at least one of the combinations selected from;
(i) up regulating TLR3 and down regulating of SHP2, and/or
(ii) up regulating TLR3, MKP and down regulating SHC,

wherein the said process comprises
(a) up regulating TLR3 by administering agonist Rintatolimod,
(b) up regulating MKP by administering agonist JWHO15,
(c) down regulating SHP2 by administering Actinomycin D, and
(d) down regulating SHC by administering 8-hydroxy-7-(6-sulfo naphthalene-2-yl)diazenyl-quinoline-5-sulfonic acid.

The said treatment may be provided to patients diagnosed with or exhibiting symptoms of cutaneous or visceral leishmaniasis.

Following examples are given by way of illustration therefore should not be construed to limit the scope of the invention.

### EXAMPLES

### Example 1

### Pathway Reconstruction/Integration

In order to reconstruct a comprehensive map of signaling processes depicting the effect of Leishmania infection on immune response, a detailed T-cell and APC interaction pathway in FIG. 1 was created after a study of existing literature and databases. Protein-protein interaction (PPI) and biochemical signal transduction data were collated from various cell signaling and PPI databases, such as KEGG, Protein Lounge, Pathway Central, Biocarta, NetPath, BIOGRID, etc. and various published research articles. Leishmania proteins were then introduced in the network and interactions of these proteins were established with existing Antigen presenting Cells (APC) molecules depending on the biological evidences. Leishmania antigenic molecules used in the present model, were as follows: lipophosphoglycan (LPG_L), GP63_L surface protease, LFAA_L and Elongation Factor (EF1_ALPHA_L), which are known to be present in almost all Leishmania species so as to construct a generalized Leishmania infection model (LFAA_L is a hypothetical molecule which the present inventors have considered in the model to show activation of ASMASE (sphingomyelinase) for production of CERAMIDE [1]; it is abbreviated for Leishmania factor activating ASMASE).

With certain modifications required to build the juxtacrine and paracrine interactions between cells, T-cell pathway reported previously (P. Ganguli, et al Temporal Protein Expression Pattern in Intracellular Signaling Cascade during T cell Activation: A Computational Study J Bioscience Vol. 40, No. 4 (2015)) was used to understand T-cell-APC cross-talks and to monitor immunological response generated during Leishmania infection. Leishmania infection was introduced in the model by establishing interaction of Leishmania antigens, known from literature and databases, with appropriate host protein molecules in the APC. Hence, to assess gene or protein expression patterns of large scale signal transduction networks under different pathological conditions, concept of discrete dynamic logical modeling approach was utilized. The pathway figure was deciphered using Cell Designer software (version 4.3). The signaling molecules (nodes) and interactions were color coded in accordance with cellular locations and their chemical nature, respectively. Also, in order to differentiate redundant Leishmania and T-cell molecules from APC molecules, names of protein/non-protein molecules were denoted with suffix ‘L’ and ‘T’ for Leishmania and T-cell, respectively.

### Example 2

### Model Formulation

The interactions of the entire network, including all important regulations between T-cell and APC, were translated into Logical equations (signifying reactions or hyperarcs) using AND, OR and NOT logical gates, in a biologically meaningful way. The model was simulated synchronously (i.e. all equations updated simultaneously) and asynchronously (i.e. random execution of equations) using BooleanNet-1.2.4 software until a steady state was reached. In this model, three functions, viz. “TH_1_response”, “TH_2_response” and “NO_response”, which reflect the type of T-cell responses were elicited and production of NO from APC in response to an infection represented by equations: Eq. 1, 2 and 3 were defined. The molecules used for defining these functions are principally the molecules involved in eliciting these responses, as reported in literature (S. Romagnani Int. J. Clin. Lab. Res. 21 (1992) 152-58).

TH_1_response*=IL2_T AND GM_CSF_T AND TNF_ALPHA_T AND IFN_GAMMA_T (Eq. 1)

TH_2_response*=IL4_T AND IL5_T AND IL6_T AND IL10_T (Eq. 2)

NO_response*=NO (Eq. 3)

### Example 3

### Properties/Features of the Reconstructed Pathway

FIG. 1 represented a simplified version of the newly reconstructed pathway diagram to provide a brief description of the entire reaction cascade. The major inter and intra cellular signaling events triggered by important molecules (e.g. MHC, CD40, IL10 etc.) of both cells and pathogen are provided for the sake of simplicity. The diagram shows juxtacrine and paracrine regulations between different cells. Leishmania antigen molecules are shown in orange. The cytoplasmic and nuclear proteins of APC and T-cell are color coded as blue and peach respectively. The target molecules that are produced as output by two cells are colored green (for protein) and deep-pink (for non-protein molecules).

The in-silico model integrates all possible inter-cellular and intra-cellular signaling events that occur between two immune cells during Leishmania invasion. The interaction of Leishmania molecules, produced from promastigote and amastigote forms, with the APC molecules are considered separately. The entire signaling network (i.e. intra and inter cellular) consists of a total of 293 nodes, which includes 82 APC molecules, 206 T-cell molecules, and 5 Leishmania related molecules, that are involved in more than 400 protein-protein interactions. The intra-cellular signaling cascades were considered for modeling APC and the major co-receptor signaling pathways were considered for modeling the T-cell. The signaling pathways consisted of cytokine pathways, TLR pathways, etc. which play a pivotal role in regulating the outcome of immune cell's functional responses. In case of APC, the pathways, which are considered in the present model, include the CD40 pathway, the Interleukin pathways (viz. IL4, IL6 and IL10), TLR pathways (TLR2, TLR3, TLR4), and the pathways involved in TNF_ALPHA, IFN_GAMMA signaling. Again in T-cell, in addition to the core TCR (T-cell receptor) mediated signaling; seven co-receptor signaling pathways (viz. CD28, CD27, LTBR, CTLA4, ICOS, PD1 and OX40), cytokine pathways (viz. IL1, IL2, IL10, IL12, TNF and IFN mediated pathways) and Calcium Release activated channel (CRAC) mediated Calcium pathway are considered. Various crosstalk reactions are also considered in the model, which depict the bi-directional regulation that exists between the two immune cells. These crosstalk reactions mainly comprise of juxtacrine signaling events stimulated directly by binding co-receptors and the ligand molecules expressed on T-cell and APC membranes, and the paracrine signaling that are mediated by the diffusible output molecules (mostly cytokines) produced by each cell. Overall 10 crosstalk interactions between the T-cell and the APC that effectively regulates the expression pattern of each otherwere considered. These include IFN_GAMMA_T, IL4_T, IL6_T, IL10_T, TNF_ALPHA_T molecules secreted from the T-cell, and IFN_BETA, TNF_ALPHA, IL12 secreted from the APC that diffuses and activates their corresponding receptor/co-receptors on their neighboring cell to trigger their downstream signaling cascades. The co-receptor ligand molecule interaction considered to be the most important in the model is the one that involves the binding of the CD40 and CD40L_T molecules (M. T. Shio et al J. Trop. Med. (2012) 819512).

The signaling events that begin at the membrane region is then considered to transduce the signal downstream to activate the major signaling pathways, such as, the MAPK (Mitogen activated protein kinases), JNK (c-Jun N-terminal kinases), NFKB (Nuclear factor kappa-light-chain-enhancer of activated B cells), JAK-STAT (system comprises of two main components: a receptor, Janus kinase (JAK) and Signal Transducer and Activator of Transcription (STAT)) cascades, which activate a series of transcription factors that eventually transcribes the output molecules. During Leishmania invasion, the antigenic molecules produced by the pathogen activate certain phosphatases (e.g. SHP1, PTP1_B, TCPTP etc.) that interfere with the signaling events of the APC. The antigen molecules considered in the network, such as LPG_L, GP63_L and EF1_Alpha, were shown to have a direct effect on the activities of the ERK1/2 and AP1 transcription factors, the former being up-regulated and the latter inhibited or degraded.

### Example 4

### Experimental Data, Reaction Initialization& Validation

Time-course microarray data for the two cells (viz. T-cell and APC) were obtained from two separate experiments from the EBI ARRAY EXPRESS database (E-GEOD: 48978 and 42088, for T-cell and APC respectively). In these microarray experiments, expression profile of activated human T-helper cell (Affymetrix HT HG-U133+ PM Array Plate) and Leishmania major infected dendritic cells (Affymetrix HG-U133 Plus 2.0 Gene Chip) were studied at discrete time-points.

The expression values at 4 time-points, i.e. 0, 2, 4, and 6 hours' time-points for T-cell and 0, 2, 4, 8 hours' time-points for dendritic cells were considered for the present analysis. This expression data were then extracted and binarized using the BOOLNET software that employs K-means clustering algorithm (C. Müssel et al Bioinformatics, 26 (2010) 1378-80). The 0^{th} hour binarized data was used to initialize all the nodes of the respective cells, with either ON or OFF depending on whether the protein shows an up-regulation or a down-regulation at the 0^{th} hour (BooleanNet Software uses TRUE and FALSE for ON and OFF respectively). The initial values of the Leishmania proteins were considered ON in the infected scenario, and OFF in the uninfected scenario. The model was then simulated using the synchronous update rule and validated by comparing the expression of 10 APC output molecules (viz. c_FOS, IFN_BETA, IL1_ALPHA, IL1_BETA, IL10, IL12, INOS, IP10, NO and TNF_ALPHA) in the infected scenario with the binarized time-course microarray data of the APC (M. A. Favila et al J. Immunol. 192 (2014) 5863-5872).

However, it should be noted that the experimental data for expression of NO molecule is considered as proportionate to the expression values of INOS of the microarray data. The model reached its steady state at the 19th time-step in the infected scenario. As a control of the experiment an uninfected scenario was also created. However, to calibrate the four experimental time points used in microarray data (i.e. 0, 2, 4, and 8 hours) with discrete time points of simulation results, logical states of the proteins up to 24 discrete time steps were considered in this analysis (after comparing the steady state values for both the experimental and simulation results). Thus, 1 hour duration of experimental data was associated by three time steps of the simulations. The temporal expression profile of the 10 output molecules were plotted till the 24th step (i.e. 8 hours of experimental data). It is to be mentioned here that since the expression of the output proteins is the best reflection of functioning of the entire signaling cascade, the validation of these previously mentioned 10 output molecules was assumed to be sufficient to demonstrate the authenticity of the entire model. The T-cell model was also validated in a similar way, by comparing time-course expression profile of the output protein molecules as obtained in synchronous simulation with the experimental data.

### Example 5

### Model Analysis and Perturbation Studies

The model was simulated asynchronously until steady state was reached to make a qualitative analysis of differences in the expression profiles and functional responses of the APC and T-cell output molecules in the infected and uninfected scenarios. The model was iterated 100 times and the average values of all simulations at each time-point were plotted for further analysis. This analysis also helped to monitor small fluctuations in the expression pattern of pathway species over time, which occurred due to the stochasticity in the execution of the pathway reactions inside the cell. In order to unravel the effect of Leishmania infection on the entire T-cell signaling cascade at the individual protein level, and then to understand the changes at the pathway level, two-tailed Mann Whitney U Test was carried out on the expression of the 163 T-cell intermediate and output molecules. This helped to identify proteins that get significantly de-regulated during the infection at 5% level of significance. Thereafter, the model was used to predict the phenotypic responses (using Eq. 1, 2 and 3) in various treatment scenarios using several gene knock-in and knock-out experiments created in-silico by trying different combinations of ON and OFF of the protein molecules using the in-built ‘boolean2.modify states’ function of the BooleanNet-1.2.4 software (I. Albert et al Source Code Biol. Med. 3 (2008) 1-8).

### Example 6

### Model Validation with Experimental Data

The temporal expression profiles of APC output molecules viz. c_FOS, IL1_ALPHA, IL1_BETA, IFN_BETA, IL10, IL12, IP10, INOS, NO, TNF_ALPHA in the infected (red) and uninfected scenarios(green) are plotted along-with binarized microarray data at 0, 2, 4 and 8 hours' time-points (black diamond) in FIG. 2. This figure depicts that expression levels of all the 10 output molecules reached the steady state values either at 1 (i.e. up-regulation) or 0 (i.e. down-regulation). Qualitative comparison of the expression values reveals that out of these 10 selected output molecules, the steady-state expression value of total 7 molecules viz. c_FOS, IL1_ALPHA, IL1_BETA, IL10, IL12, INOS and NO in the infected scenario show the exact match with the experimental observations [2]. While c_FOS and IL10 show an expression value of 1 (high expression) in the infected scenario, the other output molecules such as IL1_ALPHA, IL1_BETA, IL12, INOS and NO has an expression value of 0 (low or no expression) in the infected scenario.

Also, FIG. 2 depicts that at “4 and 8 hours” time points, c_FOS and IL10 proteins get up-regulated in simulated infected scenario, which is exactly comparable with experimentally observed expression levels in microarray data at the same time point. However, it should be noted that although the expression level of c_FOS protein at “2 hours” time point in the simulated infected scenario is not exactly matching with the experimental findings, but the infected model is able to show the down regulation of this protein between the intervals of “0 to 1 hour” time points. Both the proteins IL1_ALPHA and IL1_BETA get up regulated at “1 hour” time point and subsequently get down regulated at “6 hours” time point of the simulated infected scenario. In the experimental data, both of them get up-regulated at “2 hours” time point and get down regulated at “4 hours” and “8 hours” time points respectively. In case of IL12, it is observed from FIG. 2 that except a small time interval between 0 and 1 hour, this protein remains in the down regulated state throughout the rest of the time points. The time course microarray data of this protein also shows similar expression level except at “4 hours” time point, in which this protein shows up-regulation. Similarly, INOS and NO also show similar expression level at “2 and 8 hours” time points as compare to the experimental data. Altogether, the percentage of validation of the simulated L. major infected scenario for all the 10 selected proteins at all the three time-points i.e. 2, 4 and 8 hours are 80%, 50% and 70% respectively.

Also it can be observed that 9 out of 10 output molecules match exactly at least at two time-points. Even though in few cases, the simulation results of the expression values at a particular time point show an apparent mismatch with the experimental observation at that same time point, but the expression pattern essentially remains the same over time. It can be observed that although the time-course expression of c_FOS from the simulation results appear to be inconsistent with experimental data, i.e. down-regulation at 2 hours' and again up-regulation at 4 hours' time point, the overall dynamics of the expression essentially remains the same over time, with only a slight deviation of the expression levels (up or down) observed in the respective time points of experimental and simulation data. Such deviations are also observed in the expression dynamics of IL1_ALPHA, IL12, NO and INOS molecules. The successful validation of the expression levels of these molecules can be used as valuable indicators of the immune functions of the APC and can be used for fine-tuning of the present model to ensure its proper functioning. On the other hand, FIG. 2 also brings out the differences in the expression of the APC output molecules due to the presence of the infection. Here it is observed that even though the steady state values of two scenarios (viz. infected and the uninfected) is sometimes similar, as in cases of c_FOS, IL10 and TNF_ALPHA, the overall temporal expression pattern clearly indicates the differences emerged due to presence of antigen molecules in the model simulation. In the uninfected scenario, expression of IL10 and TNF_ALPHA remains low (in the first few hours) as compared to the infected scenario.

### Example 7

### Comparison of Uninfected and Infected Scenarios

The interference of Leishmania proteins in the signaling cascade of APC cell modulates the expression of output molecules and microbicidal activities of APC and deregulates the expression of T-cell output molecules by manipulating normal functioning of T-cell activation pathway (I. Muller, et al Annu. Rev. Immunol. 7 (1989) 561-578). Comparing expression of APC output proteins in infected and uninfected scenarios in FIGS. 3a and b, simulation results showed that invasion of Leishmania antigen molecules severely down-regulates expression of IL12, which is a potent T-cell stimulator. Simultaneously, the production of INOS and Nitric Oxide (NO) is also greatly reduced in the infected APC, thereby rendering the cell incapable of performing its microbicidal functions, and creating an immune-suppressed condition, which is favorable for continued survival of the pathogen inside APC. Besides, in FIG. 3b, production of IFN_BETA, IP10 (a chemokine) also show an up-regulation, indicating an attempt of APC to eliminate the pathogen from the system. IL1_ALPHA and IL_BETA show minor fluctuations in expression during the infection and slight down-regulation. The effect of Leishmania infection on the expression pattern of T-cell output proteins (FIGS. 3c, d) becomes evident from the fact that production of protective cytokine from the cell, such as IFN_GAMMA_T, is down-regulated during the infection, while production of interleukins, such as IL10_T, IL4_T, IL5_T and IL6_T are up-regulated, which are mostly implicated as proteins favoring Leishmania survival. These results supported by previous experimental findings also strengthen the validity of the present model to a greater extent and enhances its acceptability for further analysis.

### Example 8

### Effect of Infection on T-Cell Signaling Cascade

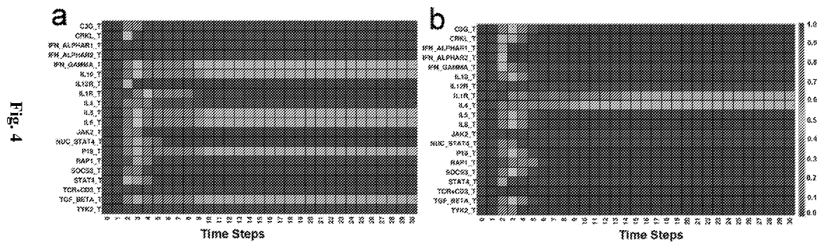

The results of Mann Whitney U test revealed that out of the expression of 62 proteins in the infected scenario that exhibit a deviation from the uninfected scenario, 20 proteins gets significantly de-regulated (p<0.05). The temporal expression profiles of these 20 proteins (FIG. 4) showed that Leishmania infection causes significant down-regulation of protective cytokines, such as IFN_GAMMA_T, and enhances synthesis of TGF_BETA_T and IL10_T from the T-cell, which contributes to decline in the immune-competency of the T-cell and formation of an immune-suppressed condition as observed during L. major infections in susceptible patients[3-6]. While activation of cytokines, such as IL4_T, IL5_T, IL6_T and receptors, IL12R_T[7]and IL1R_T[8], show fluctuations with respect to the control (uninfected scenario), certain other molecules, such as RAP1_T, P19_T, C3G_T, CRKL_T, TYK2_T and SOCS3_T, are distinctly up-regulated as a result of the infection. Also it is observed that members of the JAK-STAT pathway, such as JAK2_T and STAT4_T are down-regulated in the infected scenario (FIG. 4b).

### Example 9

### Immune Response and Immunotherapeutic Strategies

The effector molecules produced at the end of signaling processes in both T-cell and APC manifest itself in the form of a change in the phenotypic behaviors of the cell that leads to disease clearance. Through the model, these immune responses of the entire system are simulated using the functions: TH_1_response (Eq. 1), TH_2_response (Eq. 2) and NO production (Eq. 3) signifying healing response (green line), non-healing response (red line) and disease clearance (black triangular markers) respectively (FIG. 5). The pathogen load is one of the major factors, which determines the type of immune response that will be elicited during the infection. When the antigens are OFF, i.e. mimicking a situation with low pathogen load, or no infection, the Th1 and NO responses are higher as compared to the Th2 response (FIG. 5a). On the contrary, when the antigen molecules are switched ON, i.e. pathogen load is greater, a higher Th2-response is obtained (FIG. 5b). The functions i.e. Eq. 1, 2 and 3 were confirmed for their acceptability and authenticity to study the effect of conventional immunotherapeutic strategies in Leishmaniasis (i.e. IL12 and IFNγ_T), and also to predict some immunostimulatory targets to enhance anti-Leishmania immunity (Table 2). The effect of commonly practiced IL12 (FIG. 5c) and IFNγ_T (FIG. 5d) treatments were studied. Observations were made as to even though these immunostimulants enhanced the Th1 response and down-regulate the Th2 response, they failed to enhance the Nitric oxide (NO) response. Thereafter, through perturbation analysis three T-cell molecules viz. MKP_T, SHP2_T and SHC_T and two APC molecules viz. TLR3 and TLR2 that have an important role in disease clearance were identified. Single in-silico mutation study of the aforesaid molecules reveal that in MKP_T in-silico knock-in scenario (FIG. 5e), even though the Th1 response of the NO response does not increase, the Th2 response gets down-regulated as compared to the infected scenario (FIG. 5b). Knock-in mutation of the APC molecule TLR3 gives rise to an increase in NO response, although it has no significant effect on the T-cell response (FIG. 5f). In the case of in-silico knock-out mutation studies, inhibition of SHP2_T leads to up-regulation of the Th1 response and down-regulation of the Th2 response (FIG. 5g). SHC_T inhibition on the other hand, does not exhibit any significant change in T-cell or NO responses as compared to the infected scenario (FIG. 5h). However, in FIG. 5j when combinatorial therapy was used by activating TLR3 while simultaneously inhibiting SHP2_T, a better anti-Leishmania immune response was achieved. Alternatively, TLR3 knock-in when combined with SHC_T OFF (knock-out) and MKP_T ON (knock-in) can also give rise to a similar effect (FIG. 5k). Besides these combinations, it was found that if only the expression of TLR2 protein in APC was inhibited, a very high Th1 response is obtained and simultaneously the NO production is also increased drastically (FIG. 5i).

A summary of the combinatorial therapeutic strategies and their outcomes as observed from the present invention is provided in Table 2.

TABLE 2
Unique combinations of proteins used as immuno-therapeutic targets

| | | Th1 | | Th2 response | Anti- | |
|---|---|---|---|---|---|---|
| | Knock- | response up- | NO | down- | Leishmania | |
| Knock-in | out | regulation | increase | regulation | Immunity** | FIG. |
| IL12* | — | Yes | No | Yes | No | 5c |
| IFN_GAMMA_T* | — | Yes | No | Yes | No | 5d |
| MKP_T | — | No | No | Yes | No | 5e |
| TLR3 | — | No | Yes | No | No | 5f |
| — | SHP2_T | Yes | No | Yes | No | 5g |
| — | SHC_T | No | No | No | No | 5h |
| — | TLR2 | Yes | Yes | Yes | Yes | 5i |
| TLR3 | SHP2_T | Yes | Yes | Yes | Yes | 5j |
| TLR3, MKP_T | SHC_T | Yes | Yes | Yes | Yes | 5k |

*Previously known and commonly used immunotherapeutic targets.
**Anti-Leishmania immunity implies a state when Th1 and NO response is up-regulated and the Th2 response is down-regulated.

### ADVANTAGES OF THE INVENTION

The present invention provides the mechanism relating to switching between Th1/Th2 responses during Leishmania invasion in a host cell which has important implications in Leishmaniasis treatment, and hence effective regulation of this switching mechanism is important for devising a proper cure for the disease.
A treatment method employing overexpression of TLR3 in combination with inhibition of SHP2 employed in the present invention is better than the conventional IFN-γ or IL12 treatment.
The present invention addresses issues relating to Leishmania immunotherapy, such as limitations of IFN γ treatment, the reason for which IFNβ treatment is only effective at low doses and the mechanism by which the TLR molecules expressed by the APCs regulate the immune responses of the T-cell to shift the dynamics towards a higher healing Th1 response.
Large scale, intracellular T-cell signaling network is also analyzed by using this modeling technique and eventually various structural and functional properties of this network under normal and disease conditions can be studied successfully.

1. An in-silico method to identify combinations of proteins which are involved in action of a drug useful for treatment of leishmaniasis comprising the steps:
(i) reconstructing Leishmania-APC-T-cell pathway model by integrating inter-cellular and intra-cellular signalling events occurring between APC (Antigen Presenting cells) and T cell during Leishmania invasion;
(ii) simulating the Leishmania-APC-T-cell pathway model reconstructed in step (i) by AND, OR and NOT logical gates in infected and uninfected scenarios to obtain immune responses in equations selected from the group consisting of;
TH_1_response*=IL2_T AND GM_CSF_T AND TNF_ALPHA_T AND IFN_GAMMA_T (Eq. 1)
TH2_ response*=IL4_T AND IL5_T AND IL6_T AND IL10_T (Eq. 2)
NO_response*=NO (Eq. 3);
(iii) validating the immune responses as simulated in step (ii) with published literatures to confirm their acceptability and authenticity to obtain validated immune responses;
(iv) perturbing (different proteins by assigning ON/TRUE and/or OFF/FALSE to up regulate or down regulate the phenotypic functions) the validated immune responses of step (iii) to identify immuno-stimulating proteins each from APC and T-cell respectively;
(v) performing single in silico knock in/knock out mutation of the proteins identified in step (iv) to obtain in silica up-regulation/down-regulation of expression of the selected proteins;
(vi) recognizing a combination of the up-regulated/down-regulated proteins as potent immunostimulators post in silico mutation analysis in step (v) and devising their regulation to yield an effective anti-leishmania response.

2. The method as claimed in claim 1, wherein the combination of proteins comprises of three T-cell and two APC molecules.

3. The method as claimed in claim 2, wherein the T-cell molecules are selected from the group consisting of MKP_T, SHP2_T, and SHC_T.

4. The method as claimed in claim 2, wherein the APC molecules are TLR3 and TLR2.

5. The method as claimed in claim 1, wherein the Leishmania-APC-T-cell pathway model comprises 293 nodes, 82 APC molecules, 206 T-cell molecules and 5 Leishmania related molecules.

6. The method as claimed in claim 1, wherein simulating the model in step (ii) results in three phenotypic functions “TH_1_response” (Eq.1), “TH_2_ response”(Eq. 2) and “NO_response (Eq. 3).

7. The method as claimed in claim 1, wherein the in silico knock in/knock out of the selected proteins of step (v) are assigned ON/TRUE and OFF/FALSE to up regulate or down regulate the phenotypic functions as claimed in claim 6.

8. The method as claimed in claim 1, wherein the combination of immuno-stimulators of step (vi) is selected from the group consisting of Toll like receptor-2 (TLR-2) and Toll like receptor 3 (TLR-3) in Antigen presenting cells (APC's), Src Homology 2 phosphatase (SHP2) in T-cells, or Mitogen activated protein kinase phosphatase (MKP) and SHC in T-cells, for simultaneously regulating nitric oxide (NO) production, TH1 immune response and TH2 response to expedite clearance of Leishmania pathogen from an infected host cell.

9. The method as claimed in claim 8, wherein a process to increase NO production and TH1 immune response and inhibit TH2 response simultaneously in a Leishmania infected host cell comprises regulating at least one combination selected from:
(a) up regulation/stimulation of TLR3 in APC and down regulation/inhibition of SHP2 in T-cell; and
(b) up regulations/stimulation/activation of TLR3 in APC, MKP in T-cell and down regulation/inhibition of SHC in T-cell.

10. A method of using the combination of proteins as claimed in claim 2 to treat cutaneous leishmaniasis.

11. A method of using the combination of proteins as claimed in claim 2 to control Th1/Th2 immune response during leishmanial infection and to eliminate the parasite from the system.

12. A method for treating leishmaniasis comprising regulating at least one of the combinations of immune-stimulators as claimed in claim 8, wherein the combination is selected from:
(i) up regulating TLR3 and down regulating of SHP2, and
(ii) up regulating TLR3, MKP and down regulating SHC,
wherein said method comprises:
(a) up regulating TLR3 by administering agonist Rintatolimod,
(b) up regulating MKP by administering agonist JWHO15,
(c) down regulating SHP2 by administering Actinomycin D, and
(d) down regulating SHC by administering 8-hydroxy-7-(6-sulfo naphthalene-2-yl)diazenyl-quinoline-5-sulfonic acid.
